(19) United States
(12) Patent Application Publication
(10) Pub. No.: US 2008/0317804 A1
(43) Pub. Date: Dec. 25, 2008

(54) NEEM OIL LOTION PREPARATION AND WIPE

(76) Inventor: Andrew Paul O'Brien, Westhaven, CA (US)

Correspondence Address: MARGER JOHNSON & MCCOLLOM, P.C., 210 SW MORRISON STREET, SUITE 400, PORTLAND, OR, 97204, US

(21) Appl. No.: 12/143,216

(22) Filed: Jun. 20, 2008

Related U.S. Application Data
(60) Provisional application No. 60/945,862, filed on Jun. 22, 2007.
(60) Provisional application No. 60/983,512, filed on Oct. 29, 2007.
(60) Provisional application No. 61/041,292, filed on Apr. 1, 2008.

(51) Int. Cl. A01N 25/34; A61K 36/58

(52) U.S. Cl. 424/411; 424/761

(57) ABSTRACT

Provided in embodiments of the present invention is a neem oil lotion preparation impregnated in a fabric wipe to enable individualized application of an organic pesticide on human skin or a plant. In one embodiment, a wipe includes a fabric sheet impregnated with neem oil, an emulsifier, an emollient, a preservative, and water. In additional embodiments, the neem oil wipe may further be impregnated with a humectant and a fragrance.

### CROSS REFERENCE TO RELATED APPLICATIONS

This application claims the benefit of U.S. Provisional Patent Application No. 60/945,862 filed Jun. 22, 2007, U.S. Provisional Patent Application No. 60/983,512, filed Oct. 29, 2007, and U.S. Provisional Patent Application No. 61/041,292, filed Apr. 1, 2008. The contents of all the foregoing applications are hereby incorporated herein for all purposes.

### FIELD OF THE INVENTION

The present invention relates generally to a neem oil preparation, and more particularly to neem oil lotion preparation impregnated in a fabric wipe.

### BACKGROUND

Humans, animals, plants, and organisms alike must strike a balance between each other to coexist in nature. Humans may depend upon plants and animals for food. Plants and animals may develop symbiotic relationships with organisms to survive. However, certain insects, bacteria, fungi, or other organisms may become pests to humans or plants if present in over-abundance. Hence, there has generally been a need to control or remove these biological pests while preventing harm to humans, animals, plants, and beneficial organisms. A conventional technique is to apply a substance directly to a human or plant that will drive away or kill the biological pest.

Existing methods of applying a pesticide include spraying humans or plants via an aerosol or pump. However, when applying a pesticide via a spray it is difficult to control the amount and specific location of the applied pesticide. Hence, over-application or under-application issues frequently occur. Additionally, because spray techniques are not location specific in their application, unintended areas or organisms may be affected.

Pesticide products have been developed that target specific biological pests. However, these products are often toxic, especially if over-used or if applied to unintended organisms. As such, the demand for both eco-friendly products and controllable methods of applying these products has increased.

### SUMMARY

Embodiments of the present invention provide a neem oil lotion preparation, and a neem oil lotion preparation impregnated in a fabric wipe to enable individualized application of an organic pesticide on a human or plant. In one embodiment, a plant wipe includes a fabric sheet impregnated with neem oil, an emulsifier, an emollient, a preservative, and water. In additional embodiments, the plant wipe may further be impregnated with a humectant and a fragrance.

### DETAILED DESCRIPTION

Embodiments of the present invention are directed to a wipe that includes a fabric sheet impregnated with neem oil, an emulsifier, an emollient, a preservative, and water. The neem oil wipe may further be impregnated with a humectant and a fragrance.

Neem oil is a vegetable oil that is derived from the fruits and seeds of a neem plant. Neem oil can be beneficially used as an organic and bio-friendly pesticide. It has been shown to effectively repel a wide variety of biological pests while being non-toxic to mammals, birds, and beneficial insects. In addition, neem oil may be beneficial in cleansing skin and hair. It has also been shown to fight various diseases and skin conditions, as well as being therapeutic. In the lotion preparation that is impregnated in the wipe, neem oil has experimentally been shown to be most effective when included in the range of about 0.1 percent by weight to about 5 percent by weight. However, other embodiments may include neem oil in higher or lower percentages by weight.

The emulsifier helps stabilize the mixture of the other products in the lotion preparation impregnated in the wipe. A variety of emulsifiers can be used in the lotion preparation including at least one of sorbitan monostearate, lecithin, polysorbate 20, polysorbate 80, cocodemead, or the like. In experiments, polysorbate 20 included in the range of about 1 percent by weight to about 3 percent by weight for the lotion preparation has shown to be favorable. As discussed below, some embodiments include only natural ingredients. These embodiments may utilize an emulsifying agent that is derived from plant or vegetable products, such as coco derivatives, chamomile, lecithin, honey, etc.

The emollient provides lubrication and helps to reduce moisture evaporation from the lotion preparation impregnated in the neem oil wipe. The emollient also helps prevent irritation of skin. Thus, the neem oil preparation may be applied directly to the sensitive areas of skin, such as the face, in some embodiments. The emollient may also facilitate a smoother or more uniform application of the lotion preparation to the desired surface. The emollient may include at least one of cetyl alcohol, cocodithanolamide, glycerine, or the like. In experiments cetyl alcohol in the range of about 0.5 percent by weight to about 2.0 percent by weight for the lotion formula has shown to be favorable. However, cocodithanolamide, a natural coco plant derivative, may be preferable where a completely natural lotion preparation is desired.

Some of the substances used as emulsifiers or emollients may also act as a surfactant. In one embodiment, coco glucoside may be added as an emulsifier, emollient, and surfactant. Coco glucoside is an all natural ingredient that is derived from coconuts, coconut oil, and/or fruit sugar.

The preservative helps prevent spoilage and chemical changes of the lotion preparation. A wide variety of preservatives may be used in the lotion preparation, such as at least one of methyl paraben, propyl paraben, benzoic acid, and the like. In some embodiments, a combination of methyl paraben and propyl paraben is used. The experimental percent by weight ranges of these preservatives varies from about 0.05 percent by weight to about 1.0 percent by weight. In other embodiments that focus on natural ingredients, a tocopherol (Vitamin E) may be used as a preservative. In these embodiments, tocopherol acetate, tocopheryl linoleate, tocopheryl nicotinate, or the like may be utilized as a preservative.

A humectant may further be included in the lotion preparation impregnated in the neem oil wipe. Humectants generally reduce the loss of moisture in the lotion preparation and provide moisturization of skin in contact with the lotion preparation. The humectant may include at least one of glycerol, propylene glycol, sorbitol, or the like. In experiments, a combination of glycerol and propylene glycol has shown to be desirable. The experimental percent by weight ranges of these humectants varies from about 1.0 percent by weight to about 5.0 percent by weight.

A fragrance can be further added to the lotion preparation, such as lavender, lemon, almond, or the like. In experiments, a fragrance was included in the lotion preparation in the range of about 0.1 percent by weight to about 2.0 percent by weight. However, various other percent compositions may be appropriate depending on the particular fragrance used and the desired strength of the fragrance scent. Fragrances are often derived from the essential oils of plant products. Due to the concentration of essential oils, much smaller amounts of these oils may be utilized in the preparation, such as about 0.001 percent to 0.1 percent.

In addition to the elements listed above, the lotion preparation may include a variety of other ingredients. For example, the lotion preparation may include at least one of chamomile, vitamin E, or kelp extract. These additional elements may help impart color, texture, or consistency to the lotion preparation. In addition, these ingredients may help facilitate the impregnation process whereby the lotion preparation is infused in the fabric wipe. In some embodiments, these additional elements may complement one of the other elements of the lotion preparation. In one embodiment, an additional element that acts as an insect repellent may be added to prevent one or more other pests from harming a human or a plant. For example, citronella may be added to complement the neem oil in repelling harmful insects. In other embodiments, aloe may be added to the preparation to add an additional soothing ingredient when the preparation is applied to irritated skin areas. In yet other embodiments, bactericides such as phenoxyethanol and/or dehydroacetic acid may be added to the preparation to improve its cleansing properties.

Water makes up the balance of the lotion preparation that is impregnated in the neem oil wipes. It may be preferable to use purified or distilled water to prevent unwanted impurities from being included in the lotion preparation.

Table 1 below illustrates some experimental formulations of the lotion preparation impregnated in the neem oil wipes. Note that all formula compositions are given in units of percent by weight.

TABLE 1

| Ingredients | Ex #1 | Ex #2 | Ex #3 | Ex #4 | Ex #5 |
|---|---|---|---|---|---|
| Neem Oil | 1.0 | 3.0 | 2.0 | 3.0 | 5.0 |
| Emulsifier: Polysorbate 20 | 2.0 | 2.0 | 2.0 | 2.0 | 2.0 |
| Emollient: Cetyl Alcohol | 1.0 | 1.0 | 1.0 | 1.0 | 2.0 |
| Preservative: Methyl Paraben | 0.1 | 0.1 | 0.1 | 0.1 | 0.1 |
| Preservative: propyl Paraben | 0.5 | 0.5 | 0.5 | 0.5 | 0.5 |
| Humectant: Gycerol | 1.0 | 1.0 | 1.0 | 1.0 | 2.0 |
| Humectant: Propylene Gycol | 3.0 | 3.0 | 3.0 | 3.0 | 3.0 |
| Fragrance: Lavender | 1.0 | | | | 1.0 |
| Fragrance: Lemon | | 0.5 | 1.0 | | |
| Fragrance: Almond | | | | 1.0 | |
| Water | 90.4 | 88.9 | 89.4 | 88.4 | 84.4 |

In another embodiment of the neem oil wipe, the following formula is used: about 0.5 percent by weight neem oil; about 2 percent by weight glycerin or glycerol; about 0.8 percent by weight of at least one of phenoxyethanol, dehydroacetic acid, or benzoic acid; about 0.6 percent by weight coco glucoside; about 0.1 percent by weight of at least one of tocopherol (vitamin E oil) or methylisothiazolinone, 0.01 percent by weight lavender essential oil, and the remainder being water. In other embodiments, kelp may be added to the above formula. This embodiment may be advantageous in instances where all natural ingredients are desired.

The fabric wipe, which is impregnated with the lotion preparation, may include a fabric sheet including at least one layer of material that can hold a preparation and distribute the preparation when the wipe is applied to a surface, such as skin, a plant leaf, a plant stem, or other surface. Various embodiments of the fabric wipe may include woven layers of material, thicker layers of fabric, or additional layers of fabric to prevent tearing during application of the neem oil preparation on plants with rigid or sharp protrusions. Further, in embodiments utilizing a woven material, the tightness of the fabric weave may be altered to further prevent tearing. Natural, synthetic, or a combination of both natural and synthetic fibers may be used in the fabric wipe. The wipes may be structured to be disposable, that is intended for a one time use. Alternatively the wipes may be structured to be reusable. In a reusable configuration, the wipes may include absorbent fibers so that reapplication of the neem oil preparation to the wipe may be possible to impregnate the wipes with the neem oil preparation. The fabric wipes may also be structured in a variety of sizes and configurations to ease application of the neem oil preparation in a multiple application circumstance. For example, some embodiments of the wipes may be structured in a substantially rectangular shape while other embodiments may be structured as a glove-like wipe.

The lotion preparation may be prepared prior to being impregnated in the neem oil wipe. One method of preparing the lotion preparation includes heating the neem oil, emulsifier, emollient, and preservative in a first preparation while separately heating the humectant, fragrance, and water in a second preparation. In some embodiments, each of the first and second preparation may be heated to about 65° C. After each of the first and second preparation has been heated to a predetermined temperature, they are combined in mixing unit and mixed until they are combined. In some embodiments, the first and second preparation may be combined on a magnetic stirrer and agitated or mixed until the combination of the preparations reaches room temperature or about 25° C. to about 28° C.

After the lotion preparation is prepared, the lotion preparation may be impregnated in a fabric sheet. Here, the lotion preparation is caused to saturate and become infused within the fabric sheet by methods known in the art, such as soaking methods, spray-on methods, etc. In some embodiments, the neem oil preparation may be dried after being impregnated into the fabric wipes so that wipes are less messy to handle. The neem oil preparation may then be applied by first wetting the wipe with water.

Some embodiments of the present invention are advantageous over the conventional art in that they provide a biologically friendly means of applying a pesticide or pest deterrent to a specific area of a plant or group of plants. Hence, pests are deterred from harming these plants without negatively affecting the surrounding environment or the delicate ecosystem.

Other embodiments of the present invention are advantageous over the conventional art in that they provide an organic and natural preparation for use in the health and beauty industry. The impregnation of the neem oil preparation in a wipe allows for a convenient and easy manner of application without messy residues or the need of an additional application device. Single-use wipes may also be more hygienic since they are disposed of after a single use. In addition to the preparation being based largely on natural ingredients, the fabric wipes may be recyclable or bio-degradable to reduce the waste created by single-use nature of the wipes in some embodiments.

Some embodiments of the invention have been described above, and in addition, some specific details are shown for purposes of illustrating the inventive principles. However, numerous other arrangements may be devised in accordance with the inventive principles of this patent disclosure. Further, well known processes have not been described in detail in order not to obscure the invention. Thus, while the invention is described in conjunction with the specific embodiments described above, it is not limited to these embodiments or drawings. Rather, the invention is intended to cover alternatives, modifications, and equivalents that come within the scope and spirit of the inventive principles set out above.

What is claimed is:

1. A plant cleaning device comprising:
a fabric sheet including at least one layer of material; and
a lotion preparation impregnated in the fabric sheet, the lotion preparation including:
a neem oil extract present in the range of about 0.1 percent by weight to about 5 percent by weight,
an emulsifier present in the range of about 1 percent by weight to about 3 percent by weight,
an emollient present in the range of about 0.5 percent by weight to about 2 percent by weight,
a preservative present in the range of about 0.05 percent by weight to about 1 percent by weight,
a fragrance present in the range of about 0.001 percent by weight to about 2 percent by weight, and
water as the balance percentage by weight.

2. The plant cleaning device of claim 1, wherein the neem oil extract is present in the lotion preparation at about 0.5 percent by weight.

3. The plant cleaning device of claim 2, wherein the humectant is glycerin and is present in the lotion preparation at about 2 percent by weight.

4. The plant cleaning device of claim 3, wherein the preservative is at least one of a phenoxyethanol, a dehydroacetic acid, or a benzoic acid and is present in the lotion preparation at about 0.8 percent by weight.

5. The plant cleaning device of claim 4, wherein the emollient is coco-glucoside and is present in the lotion preparation at about 0.6 percent by weight.

6. The plant cleaning device of claim 5, wherein the lotion preparation further comprises at least one of a tocopherol extract or a methylisothiazolinone at about 0.1 percent by weight.

7. The plant cleaning device of claim 6, wherein the fragrance is lavender essential oil and is present in the lotion preparation at about 0.01 percent by weight.

8. The plant cleaning device of claim 7, wherein water is distilled water and is present in the lotion preparation as the balance percent by weight.

9. The plant cleaning device of claim 1, wherein the fabric sheet is configured as a glove-like wipe.

10. A lotion preparation comprising:
a neem oil extract;
an emulsifier;
an emollient; and
a preservative.

11. The lotion preparation of claim 10, wherein the neem oil extract is present in the range of about 0.1 percent by weight to about 5 percent by weight.

12. The lotion preparation of claim 11, wherein the emulsifier is present in the range of about 1 percent by weight to about 3 percent by weight.

13. The lotion preparation of claim 12, wherein the emollient is present in the range of about 0.5 percent by weight to about 2 percent by weight.

14. The lotion preparation of claim 13, wherein the preservative is present in the range of about 0.05 percent by weight to about 1 percent by weight.

15. The lotion preparation of claim 14, wherein the balance percent by weight is water.

16. A method of manufacturing a plant cleaning device, the method comprising:
heating a first preparation including a neem oil extract, an emulsifier, an emollient, and a preservative;
heating a second preparation including a humectant, a fragrance, and water;
combining the first and second preparations; and
impregnating the fabric wipe with the combined preparation.

17. The method of manufacturing a plant cleaning device according to claim 16, wherein the first and second preparation are heated to about 65° C.

18. The method of manufacturing a plant cleaning device according to claim 16, wherein impregnating the fabric wipe includes soaking the fabric wipes in the lotion preparation.

19. The method of manufacturing a plant cleaning device according to claim 18, wherein the lotion preparation is dried after being impregnated in the fabric wipes.

20. The method of manufacturing a plant cleaning device according to claim 16, wherein combining the first and second preparations includes mixing the first and second heated preparations, and cooling the combined preparation to about 25° C. to about 28° C. prior to impregnating the fabric wipe with the combined preparation.
